(19) United States
(12) Patent Application Publication
(10) Pub. No.: US 2009/0078736 A1
(43) Pub. Date: Mar. 26, 2009

(54) SURGICAL STAPLER WITH MAGNETICALLY SECURED COMPONENTS

(76) Inventor: Stephen J. Van Lue, Santa Clara, CA (US)

Correspondence Address: DAVID M QUINLAN, PC, 32 NASSAU STREET, SUITE 300, PRINCETON, NJ, 08542, US

(21) Appl. No.: 11/658,791

(22) PCT Filed: Jul. 25, 2005
(86) PCT No.: PCT/US2005/026349

§ 371 Date: Dec. 4, 2008

Related U.S. Application Data
(60) Provisional application No. 60/591,243, filed on Jul. 26, 2004.

(51) Int. Cl. A61B 17/068

(52) U.S. Cl. 227/175.1

(57) ABSTRACT

A surgical stapler includes a stapler head with a stapler cartridge from which staple blanks are ejected against an anvil with grooves that bend the staple blanks into staples that hold together tissue layers interposed between the anvil and stapler cartridge. The anvil and stapler head each includes magnetic members, at least one of which is a magnet and the other of which is either another magnet or a non-magnetized magnetically permeable member. Placing the anvil and stapler head in proximity with each other causes them to be magnetically coupled together with the anvil opposing the stapler cartridge. The anvil is then locked to the stapler head mechanically so that the tissues captured therebetween can be secured together by actuating the stapler to eject staple blanks against the anvil. In another aspect of the invention, the staple cartridge is removably secured to the stapler head by a similar magnetic arrangement.

### CROSS-REFERENCE TO RELATED APPLICATIONS

This application his application claims the benefit of U.S. provisional application No. 60/591,243, filed Jul. 26, 2004, which is incorporated herein by reference.

### BACKGROUND OF THE INVENTION

1. Field of the Invention

The present invention relates to devices for stapling tissues during surgical procedures, and more particularly, to a surgical stapler in which a stapler component, such as a stapler anvil or staple cartridge, is magnetically secured to the stapler.

2. Description of Related Art

Surgical staplers are utilized in diverse ways and in many fields of surgery. Staplers may be used to perform tissue excisions or anastomosis of luminous (hollow) structures such as intestinal tissue or vascular structures. In principle, surgical staplers work like common paper staplers, in that a staple is formed when a staple blank is forced against an anvil with grooves that bend the ends of the blank over to grasp two sheets of material.

One type of surgical stapler has an anvil that is detachable from a stapler head, which includes a staple cartridge for ejecting staple blanks. Once the anvil and stapler head are at their desired locations, the anvil is mechanically coupled to the head. The stapler includes a mechanism to align the staple blanks in the stapler head with the staple-forming grooves in the anvil prior to actuating the stapler. Staplers without a detachable anvil also have a mechanism that pre-aligns the anvil and cartridge, usually consisting of a framework of some sort upon which the anvil and cartridge are mounted to maintain proper alignment. Alternatively, another type of stapler achieves alignment of the anvil and cartridge by placing the anvil and cartridge on opposing “jaws” of a single instrument, whereby alignment is achieved when the jaws are closed. There are still other stapler configurations, but virtually all existing surgical staplers have an “open” configuration, which provides for the placement of tissues between the anvil and head, which are then closed on the tissues captured therebetween with the anvil and staple cartridge in alignment, thus permitting the tissues to be stapled. Examples of existing surgical staplers are shown in U.S. Pat. No. 4,319,576, U.S. Pat. No. 4,603,693, U.S. Pat. No. 5,104,025, U.S. Pat. No. 6,053,390, and U.S. Pat. No. 6,520,398.

In staplers employing a framework to maintain alignment of the anvil and cartridge, the size and profile of the framework can interfere with maneuvering and placement of the stapler, particularly during minimally invasive procedures. Staplers that have the anvil and cartridge on opposing jaws likewise limit the ability of a surgeon to maneuver and position the stapler during minimally invasive procedures, because adequate space must be available to allow the device to be advanced far enough through a patient access point to permit opening of the jaws. This shortcoming has been partially addressed through the development of articulating staplers, which permit deployment of the stapler at different angles to accommodate the anatomy at the target area. Nevertheless, the profiles of such staplers in their open configurations, whether employing a framework or opposing jaws, severely limit the range of deployment angles and positions afforded the surgeon.

There is a need to improve the way stapler components engage one another, particularly as applied to connecting the anvil to the staple cartridge, in all fields of surgery. Eliminating frameworks, anvil guiding rods typical in end-to-end anastomotic (EEA) staplers, and the need for jawed staplers, would be particularly advantageous. These modifications would enable a stapler anvil to be deployed independently of the stapler head/cartridge, thus providing lower profile staplers with superior maneuverability, which could thus be deployed individually from varied locations outside the patient's body.

To summarize, there has been a long-felt need for improved surgical staplers that avoid drawbacks of existing staplers, both those referred to above and those discussed below in the course of describing the present invention and preferred embodiments thereof.

### SUMMARY OF THE INVENTION

It is an object of the invention to overcome the disadvantages of the prior art by magnetically coupling stapler components together, particularly in regard to coupling a stapler anvil to a stapler head/staple cartridge.

In accordance with one aspect of the invention, a surgical stapler comprises a stapler body having at a distal end thereof a stapler head for ejecting staple blanks through a plurality of staple openings, and a separate anvil having staple-forming grooves therein for bending the ejected staple blanks into staples for fastening together tissue layers interposed between the staple head and anvil, wherein the stapler head and anvil include first and second magnetic members, respectively, at least one of the magnetic members comprising a first magnet, and the other magnetic member comprising a second magnet or a non-magnetized magnetically permeable member, the magnetic members being positioned on the stapler head and anvil for magnetically coupling the anvil to the stapler head through the tissue layers. In one embodiment, the first magnetic member is a magnet disposed on the anvil and the second magnetic member is a non-magnetized magnetically permeable member disposed on the stapler head.

In a more specific embodiment of the invention, the anvil includes an annular face with the staple-forming groves therein and the stapler head includes a staple cartridge with an annular face having staple-ejecting openings therein and a locking member for cooperating with the anvil to lock the anvil into place with the opposing faces clamping the tissue layers therebetween and with the staple-forming grooves aligned with the staple-ejecting openings. In another specific aspect of the invention, the stapler head includes a staple cartridge with an annular face having staple-ejecting openings therein, the staple cartridge being removably held magnetically in the stapler head. In yet another specific aspect of the invention, at least one of the stapler head and anvil includes a light source for transilluminating the tissue layers.

In still another aspect of the invention, a method of performing a surgical procedure comprises the steps of:
providing a surgical stapler including a stapler body having at a distal end thereof a stapler head for ejecting staple blanks through a plurality of staple openings and an anvil having staple-forming grooves therein for bending the ejected staple blanks into staples for fastening together tissue layers interposed between the staple head and the anvil, wherein the stapler head includes a locking member for cooperating with the anvil to lock the anvil into place with the tissue layers clamped therebetween and with the staple-forming grooves aligned with the staple ejecting openings, and wherein the stapler head and the anvil include first and second magnetic members, respectively, at least one of the magnetic members comprising a first magnet, and the other of the magnetic members comprising a second magnet or a non-magnetized magnetically permeable member, the magnetic members being positioned on the stapler head and the anvil for magnetically coupling the anvil to the stapler head through the tissue layers,
placing the anvil at a predetermined location in a luminous body part,
resecting the body part to provide a sealed distal end having the anvil proximate thereto and a sealed proximal end,
introducing the stapler head through a bodily orifice connected to the body part to a position proximate to a sealed proximal end of the luminous body part,
bringing the distal and proximal sealed ends of the body part into proximity to cause the first and second magnetic members to couple magnetically with the sealed ends of the body part captured between the anvil and the stapler head,
extending the locking member from the stapler head through the proximal and distal ends of the body part to the anvil to clamp the ends between the stapler head and the anvil, and
ejecting staples from the head to form an annular stapled connection between the ends of the body part.

A further aspect of the invention includes a surgical stapler comprising a stapler body having at a distal end thereof a stapler head, and a staple cartridge for ejecting staple blanks through a plurality of staple openings onto an anvil having staple-forming grooves therein for bending the ejected staple blanks into staples for fastening together tissue layers interposed between the staple head and the anvil, wherein the stapler head and the staple cartridge include first and second magnetic members, respectively, at least one of the magnetic members comprising a first magnet, and the other magnetic member comprising a second magnet or a non-magnetized magnetically permeable member, said magnetic members being positioned on the stapler head and cartridge for magnetically coupling the cartridge to the stapler head.

### BRIEF DESCRIPTION OF THE DRAWINGS

The objects of the invention will be better understood from the detailed description of its preferred embodiments which follows below, when taken in conjunction with the accompanying drawings, in which like numerals refer to like features throughout. The following is a brief identification of the drawing figures used in the accompanying detailed description.

FIG. 1 is a perspective schematic representation of an EEA stapler in accordance with one embodiment of the present invention.

FIG. 2 is a detail view of the end of the stapler head and staple-ejecting cartridge of the stapler in FIG. 1.

FIG. 3 is a detail view of the end of the stapler head and cartridge with an anvil locking rod in its operative extended position.

FIG. 4 is a detail view of the end of the anvil of the stapler in FIG. 1.

FIG. 5 is a schematic perspective view of one point in a bowel anastomotic procedure employing the stapler in FIGS. 1 to 4.

FIG. 6 depicts such a similar point in such a procedure using a prior art stapler.

### DETAILED DESCRIPTION OF THE PREFERRED EMBODIMENTS

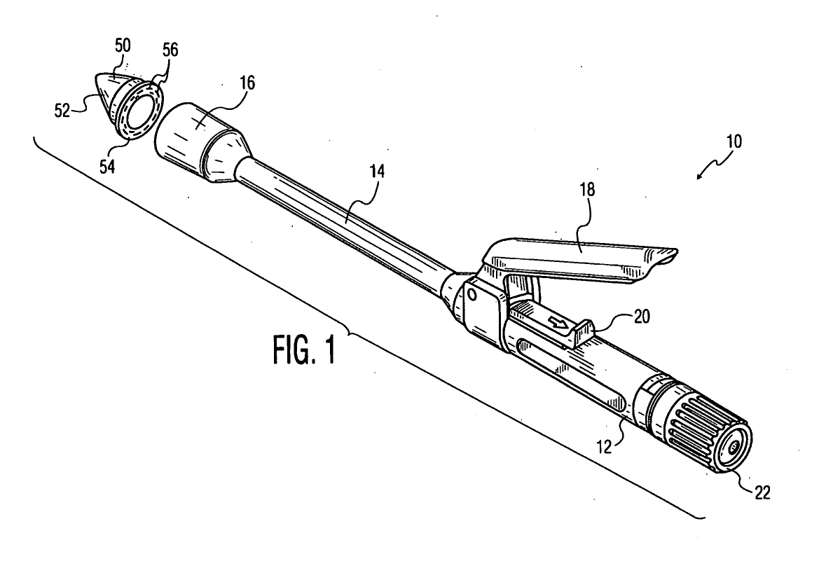

FIG. 1 schematically represents a preferred embodiment of an EEA stapler 10 in accordance with an embodiment of the present invention. As is conventional, the stapler 10 includes a stapler body with a handle 12, an elongated shaft 14, and a head 16 at a distal end of the shaft 14. The handle includes a trigger 18 and a safety 20 that prevents inadvertent actuation of the trigger. When the safety is in an “Off” position, the trigger 18 can be actuated to forcibly eject staple blanks from a staple cartridge in the head 16. A thumb screw 22 provides for manual operation of certain staple components, as discussed in more detail below. The general configuration of the stapler 10, and the internal mechanisms for ejecting staples from the head 16, is conventional. The construction of these parts is familiar to those skilled in the art, and accordingly, they are not described herein in further detail. Those construction details can be, for example, as shown in any of the patents mentioned above, the particulars of which in that regard are incorporated herein by reference.

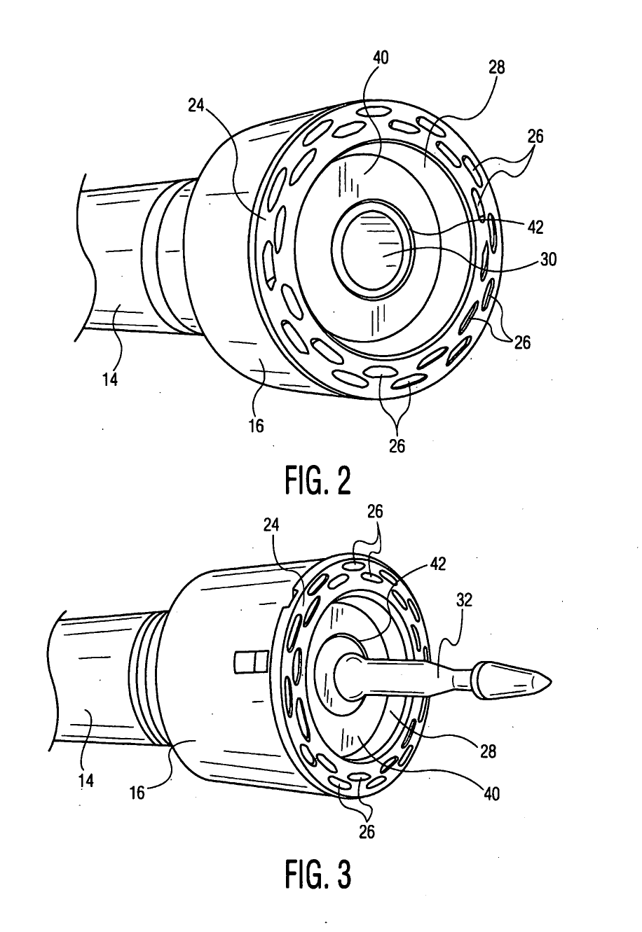

FIGS. 2 and 3 depict the distal end of the stapler head 16 in more detail. The head 16 includes a removable staple cartridge 24 that has in its face two offset annular rows of openings 26 through which staple blanks (not shown) are ejected when the trigger 18 is squeezed. Alternatively, the staple cartridge can be permanently secured to the stapler head. The head also includes a retractable cylindrical cutting blade 28 that presents a circular knife edge for cutting tissue in an anastomotic surgical procedure, as discussed in more detail below. A central lumen 30 in the head permits the axial retraction and deployment of an anvil locking rod 32. These components are also generally known, and details thereof are familiar to those of ordinary skill in the art. They may be constructed as discussed in the above identified patents, the particulars of which in these respects are also incorporated herein by reference.

FIGS. 2 and 3 also illustrate an important feature of the present invention, namely the incorporation of an annular first magnetic member 40 in the stapler head 16. The first magnetic member 40 has a generally flat face and a central lumen 42 that permits axial deployment of the anvil locking rod 32. The first magnetic member cooperates with a second such member in a removable stapler anvil 50, seen in FIG. 1.

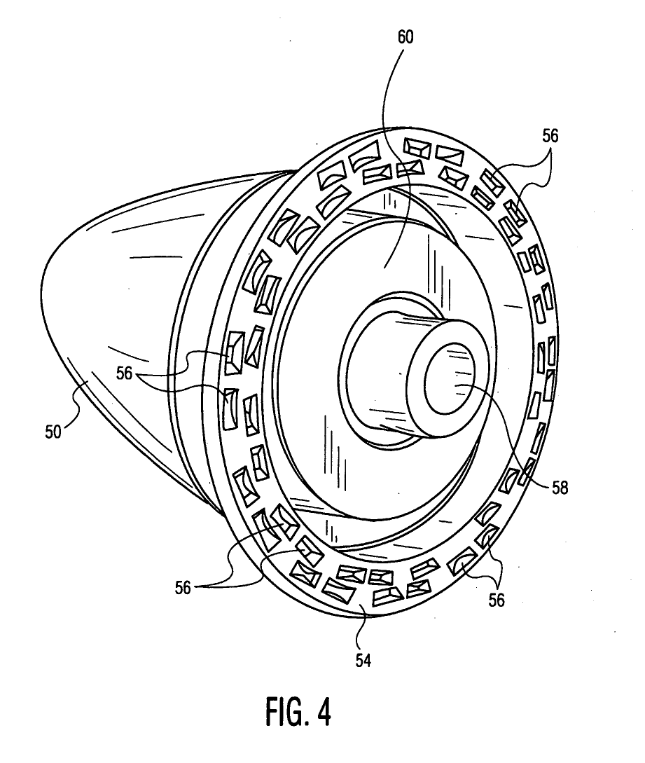

The anvil 50 has a generally conical distal end 52 to facilitate insertion during surgical procedures into luminous body structures such as intestinal sections and vascular bodies. As seen in more detail in FIG. 4, the anvil 50 according to this embodiment of the invention includes a flange 54 that presents a face that opposes the face of the staple cartridge 24 and two offset rows of staple-forming grooves 56 that align with the openings 26 in the staple cartridge when the anvil is in place opposing the face of the staple cartridge. The anvil also includes a central lumen 58 into which the anvil locking rod 32 from the staple cartridge extends when it is deployed. The anvil locking rod includes structure that securely holds the anvil in place with the grooves 56 precisely aligned with the staple-ejecting openings 26. The anvil locking rod can interact with the anvil in any suitable manner to achieve the necessary locking/alignment. One example of a mechanism used in the prior art is depicted in above-mentioned U.S. Pat. No. 5,104,025, the particulars of which in that respect are incorporated herein by reference.

When the anvil 50 is thus firmly locked to the stapler head with tissue layers captured between the faces of the anvil flange 54 and the staple cartridge 24, staple blanks ejected from the openings 26 by actuating the trigger 18 are formed into staples by the grooves 56 to secure the tissue layers together. Again, this is a conventional feature of these types of surgical staplers, and further description thereof is not necessary for one skilled in the art to understand the present invention.

FIG. 4 also illustrates an important feature of the invention, namely a second annular magnetic member 60 that cooperates with the first magnetic member 40 in the stapler head 16, as mentioned above, in a manner that is unique to the present invention. That is, an important feature of the invention is that the anvil 50 is attracted magnetically to the stapler head 16 by the placement of the first and second magnetic members 40 and 60. It will be appreciated that one of the magnetic members 40 and 60 is a magnet and the other is either another magnet or a non-magnetized, magnetically permeable material. It will be further appreciated that any suitable materials can be used for the magnet and the magnetically permeable member. Examples of materials suitable for the permanent magnet are neodymium-iron-boron (NeFeB), samarium cobalt (SmCo), and alnico (AlNiCo). NeFeB and SmCo are rare-earth magnets and are preferred because they provide a very strong magnetic force. SmCo is slightly preferred because it is more resistant to corrosion than NeFeB. Alnico can be cast or sintered and therefore can easily be made to the desired configuration and dimensions. Hard ferrite or ceramic magnets, made from a combination of either barium or strontium oxide and iron oxide can also be used. The magnetically permeable material can be a material such as cold-rolled steel or an iron-cobalt alloy (with 50% iron-50% cobalt), to name two possible materials known in the prior art. It may also be desirable to encase the permeable magnetic material and/or the magnet in a corrosion-resistant, biocompatible material.

The stapler 10 is used in anastomotic procedures by placing the anvil 50 in one section of a luminous body structure that has a closed end formed by suturing or stapling the end of the structure. For example, this could be a first bowel section. A second section of the bowel, also sutured or stapled to provide a blind opening, is accessible to the stapler head 16 transanally. By bringing the two bowel sections into proximity, by, say, conventional laparoscopic techniques, the magnetic attraction of the anvil to the stapler head holds the two blind bowel ends together. The anvil locking rod 32 is then deployed by turning the thumb screw 22, which causes the locking rod 32 to puncture the tissue layers captured between the anvil and stapler head. The anvil locking rod, when deployed, extends from the lumen 30 in the stapler head (see FIG. 3) and into the lumen 58 in the anvil. When the locking rod is fully extended, it latches to the anvil. The thumb screw 22 is then used to retract the locking rod so that the anvil is firmly clamped to the face of the stapler cartridge with the anvil grooves 56 in precise alignment with the stapler head slots 26. When the trigger 18 is actuated, the two facing bowel sections are stapled together with a circular double row of staples. The cutting blade is deployed axially by continuing to squeeze the trigger, which then slices the layers of tissue within the circle formed by the staples. The entire stapler, with the anvil secured to the head, is withdrawn from the patient, leaving the anastomosed bowel in place.

In a procedure according to this aspect of the invention, the section of the bowel that is to be removed is located by conventional laparoscopic procedures, such as direct laparoscopic visualization, palpation with a laparoscopic probe, or transmurally visualizing a previously placed intraluminal “tattoo.” The anvil 50 is then placed within the bowel away from the part of the bowel that is to be removed. One way of placing the anvil 50 is by using a separate instrument (which can be magnetic) introduced transanally and “dropping off” the anvil at the proper site within the bowel. Alternatively, after introduction into the bowel the anvil can be directed magnetically to the desired location using a magnetic “extraluminal” instrument (that is, one that couples to the anvil from outside the bowel wall), or “milked” into place by mechanically massaging the bowel wall. Once the anvil has been located as desired, it is secured using a magnetic retaining instrument that couples to the anvil across the bowel wall.

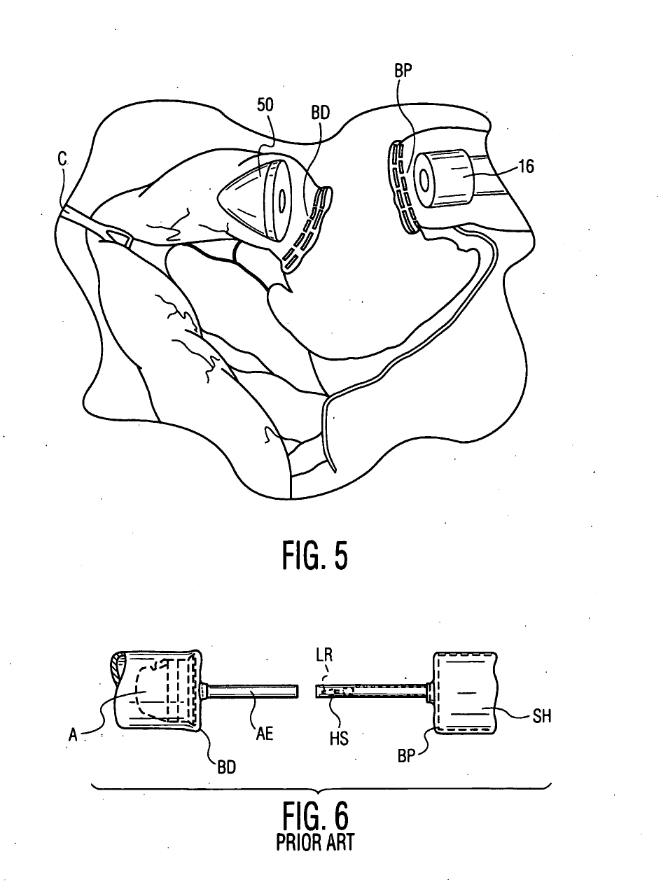

FIG. 5 illustrates in more detail one type of bowel anastomosis procedure using an EEA stapler in accordance with the present invention. The diseased bowel portion has been resected and removed using conventional techniques. For example, an in-line stapler may be used to deposit a quadruple row of staples to close off one end of the diseased bowel portion. This type of stapler includes a blade that cuts between the middle two staple rows. The other end of the diseased bowel portion is similarly stapled and cut, so that the resected, diseased bowel portion can be removed through the patient's abdominal wall (not shown). This leaves a distal sealed bowel end BD and a proximal sealed bowel end BP, each of which is sealed with a double row of staples as shown in FIG. 5. The anvil 50 is disposed within the sealed distal end BD, which can be held by the surgeon using a conventional laparoscopic instrument C. (The instrument holding the anvil 50 in place is omitted for clarity). Once the diseased bowel section is removed, the stapler head 16, introduced transanally into the bowel, can be brought proximate to the inside of the proximal sealed bowel end BP. Bringing the two bowel ends into proximity causes the magnetic members in the head 16 and anvil 50 to attract one another and tightly hold the two sealed bowel sections BD and BP in end-to-end relation.

Once the bowel sections are so located and held in place, the surgeon can turn the thumbscrew 22 to cause deployment of the anvil locking rod 32. Its sharp end punctures the proximal bowel end BP and enters the central anvil lumen 58. The locking rod is then captured within the anvil by conventional means in a manner that compresses the bowel ends together and aligns the staple-forming grooves 56 with the staple-ejecting openings 26. Squeezing the trigger 18 fires the stapler and extends the cutting blade 28 to form a lumen through the two sealed bowel ends. The stapler, with the anvil attached and the severed tissue captured between the anvil and stapler head, is then removed transanally from the patient.

FIG. 6 illustrates a similar bowel anastomosis procedure using the prior art stapler in U.S. Pat. No. 5,104,025. The diseased bowel section is removed and the bowel ends, which remain open, are brought outside the patient's body, although the procedure may also be performed entirely intracorporeally. The anvil A is then disposed in the open distal bowel end BD, which has been secured around an anvil extension AE using a “purse-string” suture. (The anvil 50 of the present invention could also be put into place in this manner, after which the bowel end BD would be sutured or stapled shut.) Likewise, the stapler head SH is disposed within the stapled or sutured proximal bowel end BP and similarly closed around a retractable head sleeve HS extending from the bowel end BP. With the head sleeve HS and anvil extension AE brought together, the anvil locking rod LR is extended to enter the anvil extension AE, which enables the anvil to be locked in place against the stapler head with the bowel sections therebetween. (Alternatively, the anvil locking rod can be extended through the sutured or stapled bowel end BP.) At that point, the procedure is similar to that discussed above. However, in a procedure employing a stapler according to the present invention, the bowel ends BD and BP can be sealed and in contact before the anvil locking rod is forced through the bowel end BP. In contrast, the prior art procedure creates the possibility that bowel contents can leak from the openings through which the anvil extension and head sleeve (or anvil locking rod) extend. Any leakage of bowel contents into the abdominal cavity of the patient during this type of procedure can have very serious consequences, leading in some cases to sepsis and death.

The present invention also has numerous additional advantages over prior art stapler configurations. In the case of staplers employing a “framework” to maintain alignment of anvil and cartridge portions, the frame adds significantly to the profile of the device. Particularly during minimally invasive procedures, it can be difficult to maneuver such a device for placement on or around the desired tissue. Staplers that have the anvil and cartridge components on opposing jaws of a single instrument also limit the ability of the surgeon to maneuver and position the device, especially in a minimally invasive procedure. Adequate space must be available to allow for the device to be advanced far enough through the patient access point to allow the device jaws to be opened. This shortcoming has been partially addressed through the development of articulating staplers, which allow the surgeon to deploy the stapler at various angles to accommodate the anatomy in the target area. Nevertheless, the profile of prior art staplers in the open configuration, whether on a framework or on opposing jaws of a single instrument, severely limits the range of deployment angles and positions.

In contrast, a stapler in accordance with the present invention permits independent deployment of the anvil and the stapler head from different anatomical locations or patient access points, and thereby maximizes the range of deployment angles and the opportunity for maneuvering the components on or around tissues. Upon locating the separated components in the desired position, the anvil and cartridge components are engaged magnetically to provide “general” alignment of the components, after which a mechanical means (the locking rod 32) may be used to lock the components together and achieve precise alignment of the staple cartridge with the anvil.

One of the advantages of the invention is that it eliminates the need for an extension shaft on the anvil to accept an anvil locking rod deployed from the stapler head. (See FIGS. 2 and 3 of U.S. Pat. No. 5,104,025 and FIG. 6.) Not only does such a shaft introduce the possibility of bowel leakage, as discussed above, it also complicates maneuvering and placement of the anvil. This is particularly true in minimally invasive procedures where the surgeon is viewing the operative field on a video monitor in only two dimensions. The present invention eliminates the anvil shaft so that the anvil may be more easily placed and guided within a luminous structure/organ to the desired location by coupling magnetically to the extra-luminal stapler head across the tissue interface. It will be appreciated that this is advantageous in procedures in the upper gastrointestinal tract in which the anvil is introduced per os (orally), as well as lower bowel procedures discussed above. This may also prove advantageous when the present invention is used in procedures involving vascular structures, where a low profile anvil (that is, one without an anvil shaft) can be more easily deployed via a catheter-based or other intraluminal delivery system.

Variations of the parts of the invention described above are possible. For example, the anvil flange 54, or even the entire anvil 50, can be a magnetic member (either a magnet or a non-magnetized magnetically permeable member), rather than introducing a separate magnetic member such as member 60. This would increase the magnetic attraction to the stapler head. By the same token, the entire staple cartridge 24 could likewise be a magnetic member. Typically, this would not interfere with the stapling action since surgical staples are normally non-magnetically permeable material, such as titanium, so as not to interact adversely with procedures such as MRI scans. In addition, the magnet used in a stapler according to the invention can be an electromagnet activated at the appropriate time during the procedure using a switch on the stapler handle.

In another variation, one or both of the anvil and stapler head includes LED's, fiber optics, or other illumination devices to facilitate placement and manipulation of the device during a procedure. Transillumination, or the transmission of light across a tissue boundary, is often utilized in minimally invasive surgery to allow an operator to discern the presence and/or location of tissue structures, such as vascular structures, within tissue boundaries. For example, a lighted catheter within a ureter allows a surgeon to identify the ureters laparoscopically by visualizing the transilluminated light as the procedure is performed, thus helping to avoid inadvertent ureter injury during laparoscopic manipulations. Incorporating lighting devices in the anvil or stapler head of the present invention will permit precise, targeted positioning and magnetic coupling of the stapler parts across a tissue boundary. That is, the projection of light from one or both of the anvil and stapler head can be used to determine the location or presence of either or both from the other side of a tissue boundary. This would be particularly advantageous in procedures using the present invention for vascular applications.

Yet another aspect of the invention involves magnetic retention of the staple cartridge in the stapler head. Many staplers are intended for single patient use, yet have the ability to be reloaded with additional staple cartridges, such staplers commonly being referred to as “multi-firing.” Once the stapler is “fired” and the staples are deployed, the device is removed from the patient, the empty staple cartridge is removed, and a new cartridge is loaded in the stapler head. This is advantageous economically, but it increases the duration of minimally invasive procedures because the stapler must be withdrawn from the patient, the empty cartridge removed, the stapler head fitted with a new staple cartridge, and the stapler reintroduced into the patient's body and repositioned before firing again. It is difficult with existing multi-firing stapler designs to reload a stapler intracorporeally (inside the body), because it is simply too difficult and time consuming in minimally invasive surgery to perform the necessary manipulations with the device inside the body. For example, one difficulty arises because the surgeon is typically viewing the procedure on a video monitor which only provides a two-dimensional viewing field and thus has no depth perception.

A removable staple cartridge retained magnetically in the stapler head, in accordance with this aspect of the invention, not only enables reloading staple cartridges intracorporeally, but also further facilitates open-body procedures. Using magnetic forces between the staple cartridge and stapler head allows for virtually effortless coupling and alignment of these components, and permits the optional use of a simple mechanical device to further secure the cartridge to the head. During a minimally invasive procedure, a separate instrument could be utilized to remove a used staple cartridge and introduce a new one. In this way, once positioned in the proper anatomical location, only minor adjustments would be needed in the overall position of the stapler before firing again. During open-body procedures, the same concept may be applied where the new staple cartridge is simply placed near the proper area on the stapler head to effect magnetic coupling. If necessary, a further mechanical locking mechanism could be used in this type of stapler. In the low-light environment of an operating room typically present during minimally invasive procedures, the ability provided by this aspect of the invention to rapidly reload staple cartridges intracorporeally, or even extracorporeally (outside the body), would be advantageous. The surgeon would be able to maintain his focus on the procedure (in the case of intracorporeal reloading), and would not have to look away from the video monitor, or readjust the position of the tissues being presented for stapling. As discussed above in connection with the anvil/stapler head aspect of the invention, either the removable stapler cartridge or the stapler head can include a magnet, while the other includes either another magnet or a non-magnetically permeable material. The materials disclosed above are suitable for this aspect of the invention, also. Those skilled in the art will be able to easily adapt known removable staple cartridge configurations, such as that disclosed in above-mentioned U.S. Pat. No. 4,603,694, the contents of which in that regard are incorporated herein by reference.

This principle of the invention may also be applied to other “multi-firing” devices that deploy tissue engaging elements from a cartridge component. For instance, a multi-firing anchor deploying device may be reloaded using the concept of the invention.

It is also known to use suction forces in all fields of surgery to provide secure attachment of surgical devices to a specific tissue. Currently, one of the more common applications of suction attaches a device to a specific tissue such that the tissue can then be manipulated, or otherwise retracted, into a desired position to facilitate the procedure. Cardiac apical retractors utilizing suction for positioning of the heart, and vascular stabilizers for coronary artery bypass grafting are two examples of such devices. Suction can be used in connection with staplers according to the present invention to stabilize or otherwise immobilize anvil or stapler head components to a tissue boundary, either alone or in combination with transillumination as described above. For example, an anvil with a magnetic member in accordance with the present invention, with a battery powered light source, is placed on one side of a tissue boundary. Then, utilizing the consequent transillumination of the tissue boundary, the position of the anvil is adjusted until the light is detected through the tissue boundary at the desired location/target, at which time suction is used to immobilize the anvil. Upon immobilization, the stapler head is brought proximate to the lighted region of the tissue boundary and immediately couples magnetically to the anvil as described above. Suction may also be used to immobilize the tissue in which the stapler head is disposed, and coupling effected by bringing the tissue with the anvil therein proximate to the immobilized tissue.

Those skilled in the art will readily recognize that the principles underlying the present invention has a wide variety of applications. A circular EEA stapler has been used for purposes of illustrating an embodiment of the invention, but that should not be taken in any way to imply that the invention is limited to such a device. The principles of the invention may be applied to any stapling device which utilizes an anvil/cartridge arrangement for staple formation, in addition to the many other applications described in the preceding text.

In that connection, only selected preferred embodiments of the invention have been depicted and described, and it will be understood that various changes and modifications can be made other than those specifically mentioned above without departing from the spirit and scope of the invention, which is defined solely by the claims that follow.

What is claimed is:

1. A surgical stapler comprising:
a stapler body having at a distal end thereof a stapler head for ejecting staple blanks through a plurality of staple openings; and
a separate anvil having staple-forming grooves therein for bending said ejected staple blanks into staples for fastening together tissue layers interposed between said staple head and said anvil;
wherein said stapler head and said anvil include first and second magnetic members, respectively, at least one of said magnetic members comprising a first magnet, and the other of said magnetic members comprising a second magnet or a non-magnetized magnetically permeable member, said magnetic members being positioned on said stapler head and said anvil for magnetically coupling said anvil to said stapler head through the tissue layers.

2. A surgical stapler as in claim 1, wherein said first magnetic member is a magnet disposed on said anvil and said second magnetic member is a non-magnetized magnetically permeable member disposed on said stapler head.

3. A surgical stapler as in claim 1, wherein:
said anvil includes an annular face with said staple-forming groves therein; and
said stapler head includes a staple cartridge with an annular face having staple-ejecting openings therein and an anvil locking member for cooperating with said anvil to lock said anvil into place with said opposing faces clamping the tissue layers therebetween and with said staple-forming grooves aligned with said staple ejecting openings.

4. A surgical stapler as in claim 3, wherein:
said anvil locking member comprises an axially deployable locking rod extendible through a head lumen in said stapler head; and
said anvil includes an anvil lumen for accepting said anvil locking rod when it is deployed from said stapler head.

5. A surgical stapler as in claim 4, wherein said first magnetic member comprises an annular ring surrounding said head lumen and said second magnetic member includes an annular ring surrounding said anvil lumen.

6. A surgical stapler as in claim 3, wherein said anvil comprises a flange including said annular face, said flange comprising said second magnetic member.

7. A surgical stapler as in claim 3, wherein said staple cartridge comprises said first magnetic member.

8. A surgical stapler as in claim 1, wherein:
said anvil includes an annular face with said staple-forming groves therein; and
said stapler head includes a staple cartridge with an annular face having staple-ejecting openings therein, said staple cartridge being removably held magnetically in said stapler head.

9. A surgical stapler as in claim 1, wherein at least one of said stapler head and said anvil includes a light source for transilluminating the tissue layers.

10. A surgical stapler as in claim 9, wherein said light source includes at least one of a light emitting diode and a fiber optic cable.

11. A surgical stapler as in claim 9, wherein said light source in said anvil is battery-powered.

12. A surgical stapler as in claim 1, wherein said first magnetic member is an electromagnet.

13. A method of performing a surgical procedure comprising the steps of:
providing a surgical stapler including a stapler body having at a distal end thereof a stapler head for ejecting staple blanks through a plurality of staple openings and an anvil having staple-forming grooves therein for bending said ejected staple blanks into staples for fastening together tissue layers interposed between said staple head and said anvil, wherein said stapler head includes a locking member for cooperating with said anvil to lock said anvil into place with the tissue layers clamped therebetween and with said staple-forming grooves aligned with said staple ejecting openings, and wherein said stapler head and said anvil include first and second magnetic members, respectively, at least one of said magnetic members comprising a first magnet, and the other of said magnetic members comprising a second magnet or a non-magnetized magnetically permeable member, said magnetic members being positioned on said stapler head and said anvil for magnetically coupling said anvil to said stapler head through the tissue layers;
placing said anvil at a predetermined location in a luminous body part;
resecting the body part to provide a sealed distal end having said anvil proximate thereto and a sealed proximal end;
introducing said stapler head through a bodily orifice connected to the body part to a position proximate to a sealed proximal end of the luminous body part;
bringing said distal and proximal sealed ends of the body part into proximity to cause said first and second magnetic members to couple magnetically with the sealed ends of the body part captured between said anvil and said stapler head;
extending said locking member from said stapler head through the proximal and distal ends of the body part to said anvil to clamp the ends between said stapler head and said anvil; and
ejecting staples from said head to form an annular stapled connection between the ends of the body part.

14. A method as in claim 13, wherein said stapler further includes an annular cutting blade for cutting a piece from the stapled ends of the body part after said stapled connection is made to reconnect the interiors of the distal and proximal ends of the body part, said method further comprising the step of removing from the bodily orifice said stapler with said anvil attached to said head and the removed piece held therebetween.

15. A method as in claim 13, wherein said anvil is placed at said predetermined location prior to resecting the body part.

16. A method as in claim 15, wherein said anvil is maneuvered into position proximate to the sealed distal end of the body part using a magnetic surgical instrument.

17. A method as in claim 16, wherein said anvil is maintained in said position by using suction to secure said magnetic instrument to another body part until said first and second magnetic members are coupled magnetically.

18. A surgical stapler comprising:
a stapler body having at a distal end thereof a stapler head; and
a staple cartridge for ejecting staple blanks through a plurality of staple openings onto an anvil having staple-forming grooves therein for bending said ejected staple blanks into staples for fastening together tissue layers interposed between said staple head and said anvil;
wherein said stapler head and said staple cartridge include first and second magnetic members, respectively, at least one of said magnetic members comprising a first magnet, and the other of said magnetic members comprising a second magnet or a non-magnetized magnetically permeable member, said magnetic members being positioned on said stapler head and said cartridge for magnetically coupling said cartridge to said stapler head.

19. A surgical stapler as in claim 18, wherein said first magnetic member is a magnet disposed on said stapler head and said second magnetic member is a non-magnetized magnetically permeable member disposed on said staple cartridge.

20. A surgical stapler as in claim 19, further comprising a separate anvil having said staple-forming grooves therein, wherein said anvil includes a non-magnetized magnetically permeable member positioned for magnetically coupling said anvil to said stapler head through the tissue layers.
